(19) United States
(12) Patent Application Publication
(10) Pub. No.: US 2015/0045650 A1
(43) Pub. Date: Feb. 12, 2015

(54) BLOOD FLOW MEASUREMENT SYSTEM BASED ON INDUCTIVE SENSING

(71) Applicant: Texas Instruments Incorporated, Dallas, TX (US)

(72) Inventors: Murali Srinivasa, Sunnyvale, CA (US); David Zakharian, San Francisco, CA (US); Preeti Rajendran, Mountain View, CA (US)

(21) Appl. No.: 14/456,694

(22) Filed: Aug. 11, 2014

Related U.S. Application Data
(60) Provisional application No. 61/864,347, filed on Aug. 9, 2013.

(51) Int. Cl. A61B 5/0265; A61B 5/00

(52) U.S. Cl. CPC A61B 5/0265; A61B 5/6803; A61B 5/725
USPC 600/409

(57) ABSTRACT

An inductive sensing system is adapted for noninvasive measurement of blood flow through a blood vessel. A resonant sensor is disposed in proximity to the blood vessel, and includes a coil resonator that generates a magnetic field within a sensing area that includes the blood vessel. The resonator changes resonance state based on changes in a flow of blood hemoglobin through the sensing area. The IDC unit establishes an IDC control loop, including the resonator as a loop filter, that provides feedback resonance control of the resonator to maintain a resonant frequency state (steady state oscillation) representative of blood hemoglobin flow through the sensing area. A feedback resonance control signal provides sensor data corresponding to the resonant frequency state as representative of blood flow through the sensing area. In one embodiment, the IDC control loop is implemented as a negative impedance control loop, controlling negative impedance to counterbalance resonant impedance.

### CROSS-REFERENCE TO RELATED APPLICATIONS

Priority is claimed under USC§119(e) to: (a) U.S. Provisional Application 61/864,347 (Texas Instruments docket TI-74137PS), filed 9 Aug. 2013.

### BACKGROUND

1. Technical Field

This Patent Document relates generally to noninvasive measurement of blood flow, such as for measuring heart rate.

2. Related Art

Blood hemoglobin is characterized by heme protein molecules/groups that include an Fe2+ ion. Blood hemoglobin flowing through blood vessels can be characterized as a flow of Fe2+ ions which fluctuate (pulses) based on heart rate.

### BRIEF SUMMARY

This Brief Summary is provided as a general introduction to the Disclosure provided by the Detailed Description and Figures, summarizing some aspects and features of the disclosed invention. It is not a complete overview of the Disclosure, and should not be interpreted as identifying key elements or features of the invention, or otherwise characterizing or delimiting the scope of the invention disclosed in this Patent Document.

The Disclosure describes apparatus and methods adaptable for noninvasive measurement of blood flow through a blood vessel within a body. The methodology is operable in an inductive sensing system, including a resonant sensor adapted for disposition external to the body, in proximity to the blood vessel, where the resonant sensor includes a resonator with a resonator coil, and is characterized by a resonance state (resonator oscillation amplitude and resonator frequency), including a resonant frequency state (steady-state oscillation), and where the resonator operable to generate, from the resonator coil, a magnetic field within a sensing area that includes the blood vessel, the resonator changing resonance state based on changes in blood flow as characterized by a flow of blood hemoglobin through the sensing area.

Aspects and features of the methodology for noninvasive blood flow measurement includes establishing an IDC control loop, including the resonator as a loop filter, operable to maintain the resonator resonance state at the resonant frequency state representative of blood hemoglobin flow through the sensing area, including: (a) determining changes in resonance state of the resonator relative to a resonant frequency state representative of blood hemoglobin flow through the sensing area, and generating a corresponding resonance control signal; and (b) adjusting the resonator resonance state in response to the resonance control signal to maintain the resonator resonance state at the resonant frequency state. Sensor data is provided corresponding to the resonance control signal, such that the output sensor data corresponds to the resonant frequency state as representative of blood flow through the sensing area.

Other aspects and features of the methodology include: (a) configuring the resonant sensor with an axial coil, such that the blood vessel extends axially within the coil, and such that the sensing area is in the axial region of the axial coil; (b) configuring the resonant sensor with a planar coil, such that the sensing area is spaced from, and substantially orthogonal to a longitudinal axis of, the planar coil, and the magnetic field within the sensing area is characterized by magnetic field vector magnitudes that intersect the sensing area with a normal component that is substantially greater than an associated tangent component.

In other aspects and features of the methodology: (a) determining changes in resonance state of the resonator is accomplished by determining changes in resonator oscillation amplitude, and generating, as the resonance control signal, a negative impedance control signal; (b) adjusting the resonator resonance state is accomplished by presenting to the resonator a negative impedance controlled in response to the negative impedance control signal, so as to maintain the resonator resonance state at a resonant frequency state representative of blood hemoglobin flow through the sensing area; such that (c) determining changes in resonator oscillation amplitude, and presenting to the resonator a controlled negative impedance, establishes a negative impedance control loop operable to control the negative impedance presented to the resonator to counterbalance a resonant impedance of the resonator, thereby maintaining the resonant frequency state; such that (d) the output sensor data corresponds to the negative impedance control signal, and thereby the negative impedance required to counterbalance resonator resonant impedance as representative of blood flow through the sensing area.

Other aspects and features of the invention claimed in this Patent Document will be apparent to those skilled in the art from the following Disclosure.

### BRIEF DESCRIPTION OF THE DRAWINGS

FIGS. 1-4 illustrate example embodiments of resonant inductive sensors adapted for noninvasive sensing of blood flow through blood vessels, using sensor arrangements/structures with axial and planar sensing coil configurations:

FIG. 1 illustrates an example embodiment of a finger ring incorporating an axial sensing coil.

FIG. 2 illustrates an example embodiment of a wrist band incorporating an axial sensing coil.

FIG. 3 illustrates an example embodiment of a wrist band incorporating a planar sensing coil.

FIG. 4 illustrates an example embodiment of a sensor structure incorporating a planar sensing coil, adapted for attachment to an arm of a pair of spectacles that when worn locates the sensor in proximity to a temporal region of the head.

FIG. 5A illustrates an axial sensing coil in which a magnetic field sensing area is within the axial region of the sensing coil.

FIGS. 5B/5C illustrates a planar sensing coil in which a magnetic field sensing area is beneath, and orthogonal to a longitudinal axis of, the coil, characterized by magnetic flux (magnetic field vectors) through the sensing area.

FIG. 5D illustrates an example waveform representation of heart rate as measured through noninvasive inductive sensing of blood flow through a blood vessel.

FIG. 6 is an example functional illustration of an inductive sensing system for noninvasive inductive sensing of blood flow, including a resonant sensor adaptable for location in proximity to a blood vessel, and an inductance-to-digital conversion (IDC) unit establishing a closed IDC control loop, incorporating the resonator as a loop filter, that controls power injected into the resonator to maintain a resonant frequency state (steady-state oscillation), and provides an IDC control loop output as sensor data representative of the flow of blood hemoglobin through the sensing area established by the resonant sensor.

FIG. 7 illustrates an example embodiment of an IDC unit that implements an IDC control loop as a negative impedance control loop, regulating resonator oscillation amplitude to a constant level corresponding to a resonant frequency state, and providing sensor data outputs that characterize resonance state (including the resonant frequency state) based on either or both resonator oscillation amplitude (corresponding to resonator impedance) and resonator frequency (corresponding to resonator inductance).

### DETAILED DESCRIPTION

This Description and the Figures disclose example embodiments and applications that illustrate various features and advantages of the invention, aspects of which are defined by the Claims. Known circuits, functions and operations are not described in detail to avoid unnecessarily obscuring the principles and features of the invention.

In brief overview, an inductive sensing system is adapted for noninvasive measurement of blood flow. An example application is measuring heart rate. The inductive sensing system includes a resonant sensor and an inductance-to-digital (IDC) conversion unit.

The resonant sensor is disposed in proximity to one or more blood vessels, and includes the resonant sensor, including a resonator with a resonator coil. The resonator is characterized by a resonance state (resonator oscillation amplitude and resonator frequency), including a resonant frequency state in which the resonator is maintained at resonance (steady-state oscillation). The resonator (resonator coil) generates a magnetic field within a sensing area that includes the blood vessel, and is operable in a resonant frequency state representative of a flow of blood hemoglobin through the sensing area.

The IDC unit is configured to convert a change in resonance state into sensor data representative of the flow of blood hemoglobin through the sensing area. The IDC unit includes: (a) resonator control circuitry configured to adjust resonator resonance state in response to a resonance control signal; and (b) IDC loop circuitry configured to determine changes in resonance state relative to a resonant frequency state representative of blood hemoglobin flow through the sensing area, and generate the resonance control signal. The resonator control circuitry and the IDC loop circuitry establish an IDC control loop, including the resonator as a loop filter, operable to maintain the resonator resonance state at the resonant frequency state representative of blood hemoglobin flow through the sensing area. Sensor data output circuitry is configured to output sensor data corresponding to the resonance control signal, such that the output sensor data corresponds to the resonant frequency state as representative of blood flow through the sensing area.

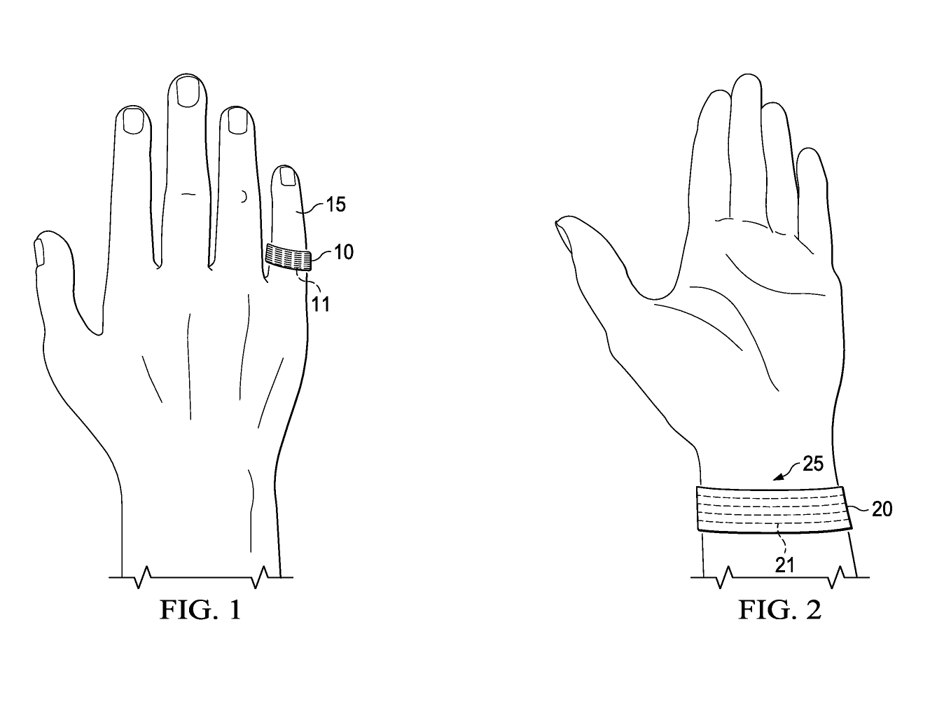

FIGS. 1-4 illustrate example embodiments of resonant inductive sensing adapted for noninvasive sensing of blood flow through blood vessels. A resonant sensor arrangement/structure incorporates a resonator coil using either an axial or planar sensing coil configuration, in parallel or series with a resonator capacitor (and characterized by a resonator impedance). The resonator arrangement/structure is adapted to locate the resonator coil in proximity to one or more blood vessels through which blood flow is to be measured.

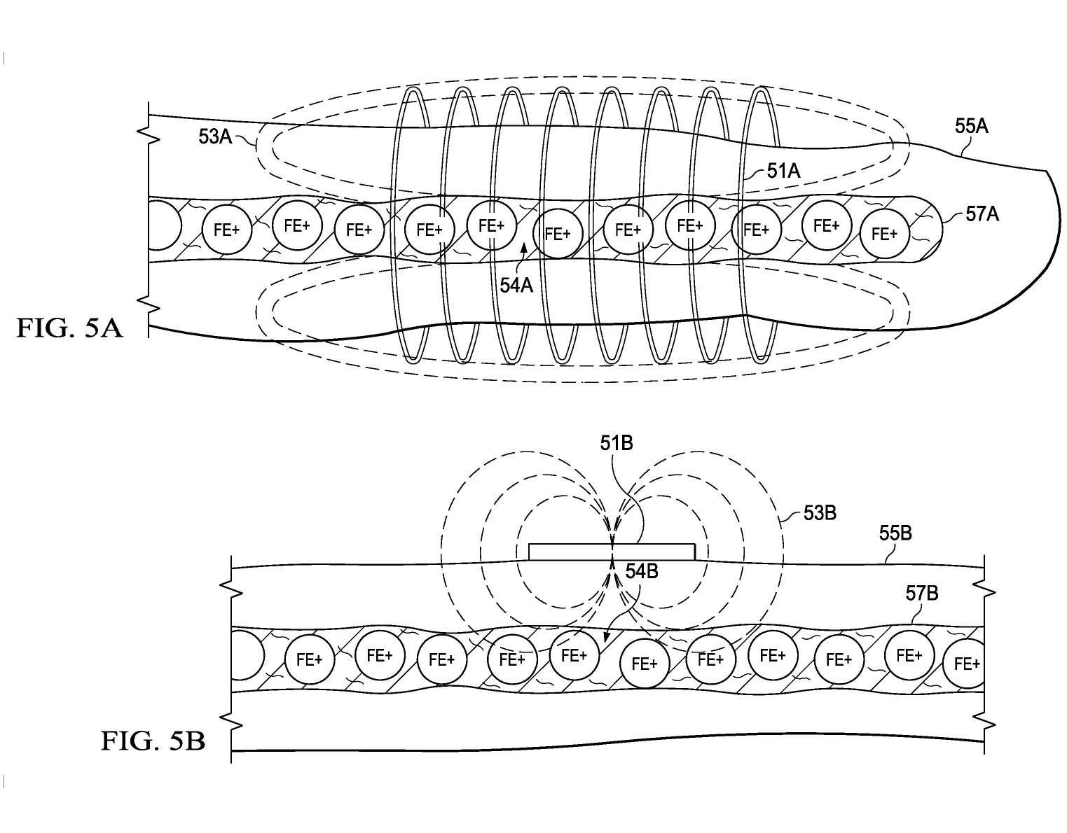

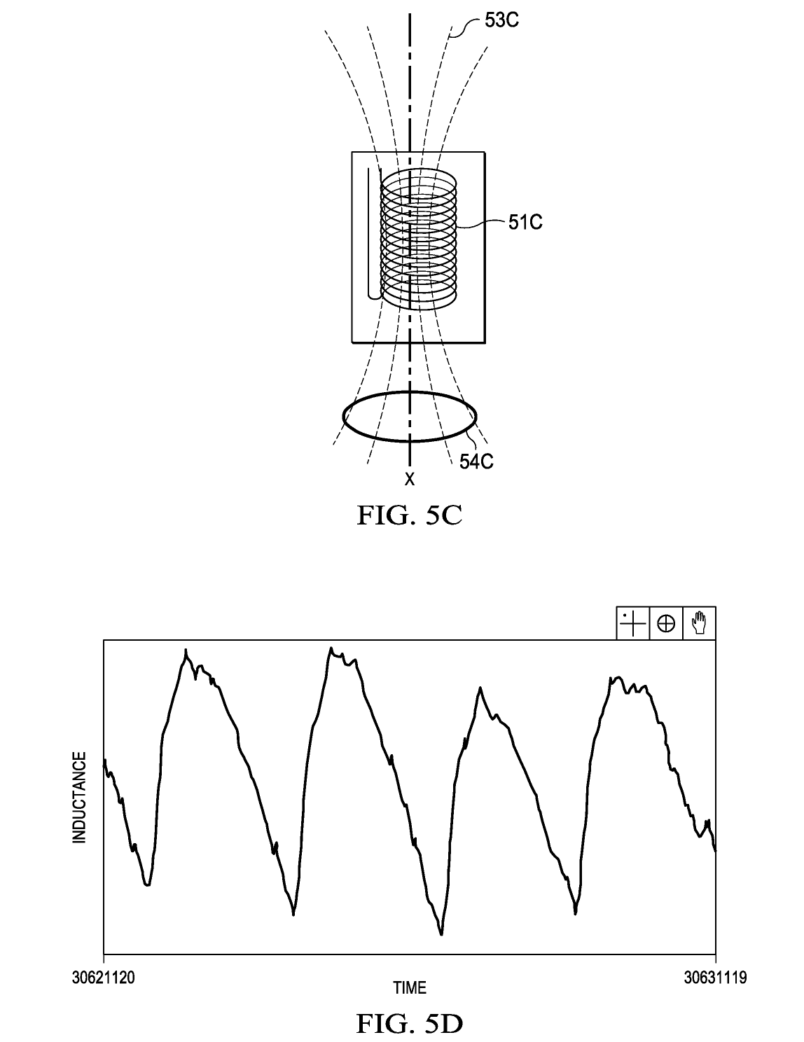

Operating in a resonant frequency state (a resonance state with steady-state oscillation), the resonant sensor (resonator) generates a time-varying magnetic field. As described further in connection with FIGS. 5A/B/C, by configuration and design, the resonant sensor establishes a sensing area of concentrated magnetic flux that includes a portion of a blood vessel.

Blood hemoglobin flowing through blood vessels can be characterized as a flow of Fe2+ ions through the sensing area. The concentration of Fe2+ ions within the sensing area corresponds to blood volume, which fluctuates (pulses) based on heart rate.

The flow of blood hemoglobin through the sensing area of a resonant sensor affects the magnetic field generated by the resonant sensor within the sensing area. This affect is manifested as a change in a resonance state (resonator oscillation amplitude and resonator frequency), and more specifically, to a change in resonant frequency state (steady-state oscillation). This change in resonant frequency state correlates to a change in resonant impedance as can be measured by an change resonator excitation power required to maintain a resonant frequency state (steady state oscillation), counterbalancing the resonant impedance.

FIG. 1 illustrates an example embodiment of a finger ring incorporating an axial sensing coil. Ring 10 incorporates an axial sensing coil winding 11 with an axial sensing area (FIG. 5A). With ring 10 disposed around finger 15, the axial sensing coil winds around one or more target blood vessels in the finger, such that a target blood vessel extends through the axial sensing area.

FIG. 2 illustrates an example embodiment of a wrist band incorporating an axial sensing coil. Band 20 incorporates an axial sensing coil winding 21 with an axial sensing area (FIG. 5A). With band 20 disposed around wrist 25, the axial sensing coil winds around one or more target blood vessels in the wrist, such that a target blood vessel extends through the axial sensing area.

FIG. 3 illustrates an example embodiment of a wrist band incorporating a planar sensing coil. Wrist band 30 incorporates a planar sensing coil 31 with a lateral sensing area (FIGS. 5B/C). With band 30 disposed around wrist 35, planar sensing coil 31 is located in proximity to one or more blood vessels in the wrist, such that a target blood vessel extends through the lateral sensing area.

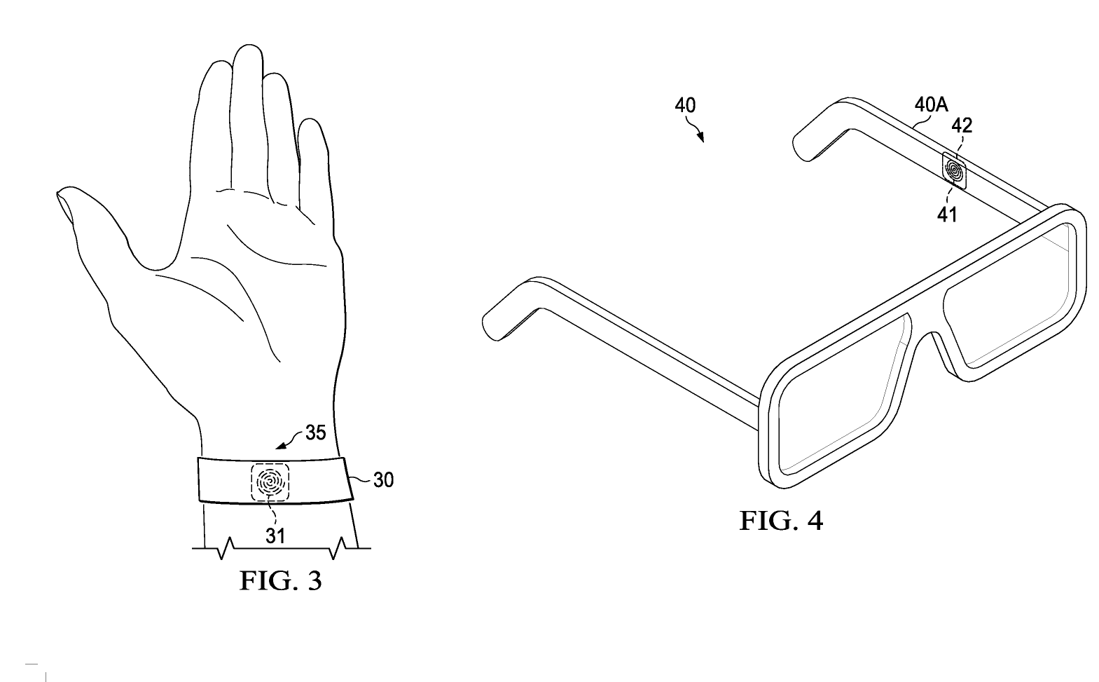

FIG. 4 illustrates an example embodiment of a sensor structure incorporating a planar sensing coil with a lateral sensing area (FIGS. 5B/C), adapted for attachment to a wearable appliance or accessory, in proximity to one or more target blood vessels. For the example embodiment, a sensor structure 42 incorporating a planar sensing coil 41 is adapted for attachment to an arm 40A of a pair of spectacles 40. When the spectacles are worn, the sensor 41/42 is located in proximity to a temporal region of the head, with a sensing area that includes one or more temporal blood vessels.

FIG. 5A illustrates an axial sensing coil 51A, in which a magnetic field sensing area 54A is in within the axial region of the sensing coil. Under excitation, axial sensing coil 51A generates a magnetic field 53A. A magnetic sensing area 54A of concentrated magnetic flux is defined within an axial region of coil 51A.

Referring also to FIG. 1, when axial sensing coil 51A is disposed around, for example, a finger 55A, blood vessels 57A extend axially through the sensing area 54A within the coil. Correspondingly, blood hemoglobin flow 57A is through sensing area 54A.

FIGS. 5B/5C illustrates a planar sensing coil 51B/C in which a magnetic field sensing area 54B/C is beneath, and orthogonal to a longitudinal axis of, the coil. Under excitation, planar sensing coil 51B/C generates a magnetic field 53B/C.

Referring in particular to the representation in FIG. 5C, a magnetic sensing area 54C is spaced from, and substantially orthogonal to a longitudinal axis of, planar coil 51C. The magnetic field 53C within sensing area 54C is characterized by magnetic field magnitude vectors that intersect the sensing area with a normal component that is substantially greater than an associated tangent component, establishing a sensing area of concentrated magnetic flux.

When planar sensing coil 51B is incorporated into, for example, a wrist band (FIG. 3) or attached to spectacles (FIG. 4), the coil is disposed on or over skin 55B, in proximity to blood vessel 57B. Blood vessel 57B extends through sensing area 54B. Correspondingly, blood hemoglobin flow 58B is through sensing area 54B.

FIG. 5D illustrates an example waveform representation of heart rate as can be measured by noninvasive inductive sensing according to the invention.

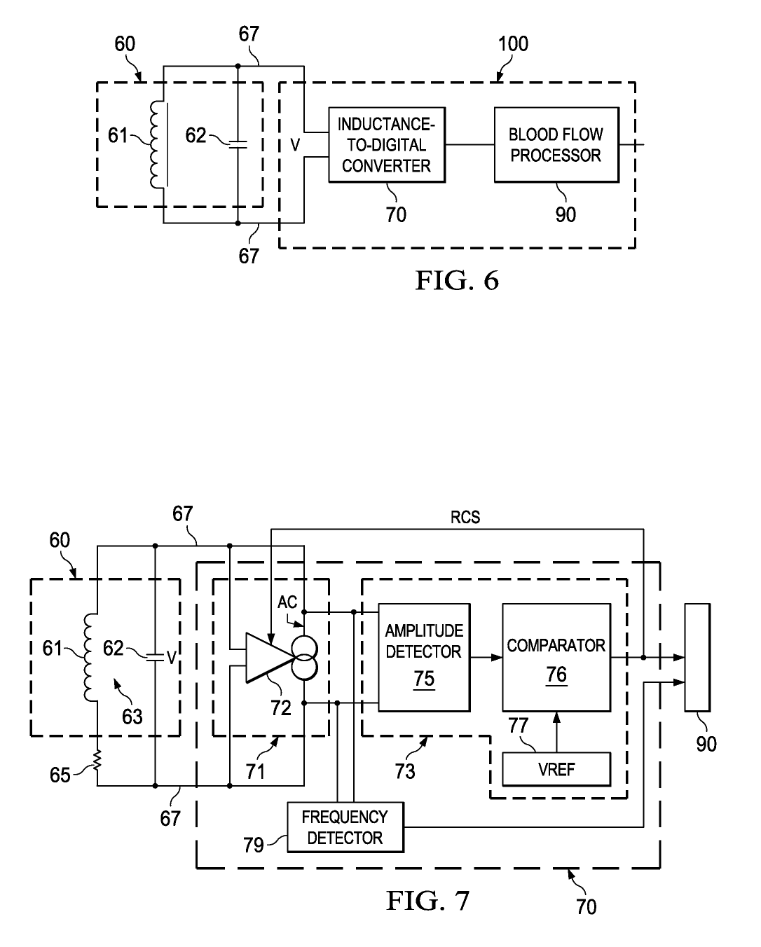

FIG. 6 is an example functional illustration of an inductive sensing system adaptable for noninvasive measurement of blood flow through a blood vessel. The inductive sensing system includes a resonant sensor 60 and a sensor converter/processor 100. Sensor converter/processor 100 includes an inductance-to-digital conversion (IDC) unit 70, and a data processor 90 operable in the example application as a blood flow processor.

Resonant sensor 60 is adaptable for noninvasive location in proximity to a blood vessel. Resonant sensor 60 and IDC 70 are configured to establish a defined sensing area (FIGS. 5A/B) that encompasses a target blood vessel, and capture/convert sensor data representative of blood flow through the sensing area. IDC 70 establishes an IDC control loop, that includes resonant sensor 60 (resonator 63) as a loop filter, controlling the excitation power input to the resonant sensor, such that the IDC control loop output provides the sensor data representative of blood flow through the sensing area.

Application-specific design considerations include skin depth, resonant frequencies and IDC resolution. In particular, skin depth (or penetration depth, or tissue depth) involves design criteria in the context of, for example: (a) tissue depth (tissue between the skin surface and a target blood vessel), which must be penetrated by the magnetic field from the resonator, so that resonator frequency is preferably low enough that skin depth is greater than tissue depth, and (b) sensing area is concentrated within the blood vessel (i.e., within the sensing range of the resonant sensor).

Resonant sensor 60 includes a resonator 63 (tank circuit) with a resonator coil 61 and a parallel resonator capacitor 62. Resonator coil 61 and resonator capacitor 62 can be configured as a series resonator.

Resonator 63 is operable to generate, from resonator coil 61, a magnetic field within a sensing area that includes one or more target blood vessels. Resonator 63 is characterized by a resonance state (resonator oscillation amplitude and resonator frequency), including a resonant frequency state (steady-state oscillation) representative of a flow of blood hemoglobin through the sensing area. That is, resonator 63 changes resonance state (resonance frequency state) based on changes in blood flow as represented by a flow of blood hemoglobin through the sensing area.

IDC 70 (IDC control loop) operates to convert resonator resonance state into sensor data representative of blood flow through the sensing area. IDC 70 is interfaced to resonant sensor 60 (resonator 63) through a wiring assembly 67, incorporating resonator 63 within the IDC control loop. IDC 70 can be located remote from resonant sensor 60.

The IDC control loop, including resonator 63 as a loop filter, controls excitation power injected into resonator 63 to maintain the resonant frequency state (steady-state oscillation). The resonant frequency state, then, is representative of blood hemoglobin flow within the sensing area (i.e., within the magnetic field established by resonant sensor 60 in the sensing area), such that a resonance control signal from the IDC control loop provides sensor data representative of the resonant frequency state, and therefore representative of blood flow within the sensing area.

The IDC control loop can be implemented with resonator control circuitry and the IDC loop circuitry. The resonator control circuitry is configured to adjust resonator resonance state in response to a resonance control signal. The IDC loop circuitry is configured to determine changes in resonance state relative to a resonant frequency state representative of blood hemoglobin flow through the sensing area, and in response generate the resonance control signal.

IDC 70 can include sensor data output circuitry configured to output sensor data corresponding to the resonance control signal, such that the output sensor data corresponds to the resonant frequency state as representative of blood flow through the sensing area. The sensor data (resonance control signal) is provided to data processor 90, operable in the example application as a blood flow processor.

FIG. 7 illustrates an example implementation of an IDC unit 70. In this example implementation, a closed IDC control loop is implemented as a negative impedance control loop that incorporates the resonator as a loop filter. The negative impedance control loop regulates resonator oscillation amplitude (at the resonance frequency) to a constant level by controlling the injection of excitation power into the resonator, counterbalancing resonant impedance. The sensor data output of the negative impedance control loop corresponds, in this implementation, to resonant impedance.

IDC 70 is coupled to resonator 63 including resonator coil 61 and resonator capacitor 62. A resistor 65 in series with resonator coil 61 represents a resonant impedance for resonator 63 (i.e., impedance at a resonant frequency state).

IDC 70 is implemented with a negative impedance circuit 71 and an impedance control circuit 73. Negative impedance circuit 71 presents a controlled negative impedance to resonator 63. Negative impedance is controlled based on resonator oscillation amplitude as a measure of the resonance state of resonator 63. Impedance control circuit 73 generates a resonance control signal RCS that controls negative impedance from negative impedance circuitry 71 to maintain a resonant frequency state, corresponding to the excitation power required to maintain steady-state oscillation.

Negative impedance circuit 71 and impedance control circuit 73 establish a negative impedance control loop that includes sensor resonator 63 as a loop filter. The negative impedance control loop is operable to control negative impedance presented to resonator 63 to counterbalance the (positive) resonant impedance of resonator 63 (represented by series resistance 65 or an equivalent parallel resistance), and maintain a resonance frequency state.

For the example implementation, negative impedance circuit 71 is implemented as a transconductance amplifier 72, configured as a controlled negative impedance. Impedance control circuitry 73 is implemented as an amplitude control circuit that detects changes in resonator oscillation amplitude as representing changes in resonance state, and provides the feedback RCS resonance control signal. The RCS resonance control signal is input to transconductance amplifier 72 to control negative impedance, and thereby control the amount of excitation power supplied to resonator 63 to counterbalance changes in resonant impedance, and maintain a resonant frequency state (steady-state oscillation).

Impedance control circuit 73 includes an amplitude detector 75 and a comparator output circuit 76. Amplitude detector 75 determines resonator oscillation amplitude. A comparator output circuit 76 compares resonator oscillation amplitude from amplitude detector 75 to a reference amplitude 77, and generates the resonance control signal RCS.

Referring to FIGS. 6 and 7, for the example implementation in FIG. 7, negative impedance circuit 71 comprises resonator control circuitry configured to present to the resonator a negative impedance controlled in response to a negative impedance control signal, so as to maintain the resonator resonance state at the resonant frequency state representative of blood hemoglobin flow through the sensing area, and impedance control circuit 73 comprises IDC loop circuitry configured to determine changes in resonance state relative to such resonant frequency state based on changes in resonator oscillation amplitude, and generate the negative impedance control signal. Negative impedance circuit 71 and the impedance control circuit 73 establish a negative impedance control loop (IDC control loop), including resonator 63 as a loop filter, operable to control the negative impedance presented to the resonator to counterbalance a resonant impedance of the resonator, thereby maintaining the resonant frequency state. The negative impedance control signal constitutes output sensor data that corresponds to the negative impedance required to counterbalance resonator resonant impedance as representative of blood flow through the sensing area.

Thus, for this example implementation of an IDC unit based on a negative impedance control loop, changes in blood hemoglobin flow through the sensing area are detected as changes in resonator oscillation amplitude corresponding to changes in the (positive) resonant impedance of the sensor resonator. The negative impedance control loop operates to adjust negative impedance to counterbalance the resonant impedance of the sensor resonator, maintaining a substantially constant resonator oscillation amplitude, corresponding to a resonant frequency state (steady-state oscillation).

The example implementation of IDC 70 includes a frequency detector circuit that measures the resonator oscillation frequency for resonator 63. For example, the frequency detection circuit can be implemented with a frequency counter. Resonator oscillation frequency can be used to determine inductance for resonator 63 (resonator coil 61), which also changes based on changes in resonance state.

IDC 70 provides separate sensor data output for resonator oscillation amplitude and resonator oscillation frequency: resonator oscillation amplitude is provided as the RCS negative impedance control signal from impedance control circuit 73, and resonator oscillation frequency is provided by frequency detector circuit 79. These sensor data outputs are provided to sensor data (blood flow) processor 90, for use in blood flow processing (such as heart rate).

The Disclosure provided by this Description and the Figures sets forth example embodiments and applications, including associated operations and methods, that illustrate various aspects and features of the invention. These example embodiments and applications may be used by those skilled in the art as a basis for design modifications, substitutions and alternatives to construct other embodiments, including adaptations for other applications, Accordingly, this Description does not limit the scope of the invention, which is defined by the Claims.

1. An inductive sensing system adapted for noninvasive measurement of blood flow through a blood vessel within a body, comprising:
a resonant sensor disposed in proximity to the blood vessel, external to the body;
the resonant sensor including a resonator with a resonator coil, the resonator characterized by a resonance state (resonator oscillation amplitude and resonator frequency), including a resonant frequency state (steady-state oscillation),
the resonator operable to generate, from the resonator coil, a magnetic field within a sensing area that includes the blood vessel, and
the resonator operable in a resonant frequency state representative of a flow of blood hemoglobin through the sensing area; and
an inductance-to-digital conversion (IDC) unit coupled to the resonant sensor, and configured to convert a change in resonance state into sensor data representative of the flow of blood hemoglobin through the sensing area, including:
resonator control circuitry configured to adjust resonator resonance state in response to a resonance control signal; and
IDC loop circuitry configured to determine changes in resonance state relative to the resonant frequency state representative of blood hemoglobin flow through the sensing area, and generate the resonance control signal;
the resonator control circuitry and the IDC loop circuitry establishing an IDC control loop, including the resonator as a loop filter, operable to maintain the resonator resonance state at the resonant frequency state representative of blood hemoglobin flow through the sensing area; and
sensor data output circuitry configured to output sensor data corresponding to the resonance control signal, such that the output sensor data corresponds to the resonant frequency state as representative of blood flow through the sensing area.

2. The system of claim 1, wherein the resonant sensor is configured with an axial coil, such that the blood vessel extends axially within the coil, and such that the sensing area is in the axial region of the axial coil.

3. The system of claim 2, wherein the axial coil is incorporated in one of a finger ring in which the blood vessel is within a finger, and a wrist band in which the blood vessel is within a wrist.

4. The system of claim 1 wherein the resonant sensor is configured with a planar coil, such that the sensing area is spaced from, and substantially orthogonal to a longitudinal axis of, the planar coil, and the magnetic field within the sensing area is characterized by magnetic field vector magnitudes that intersect the sensing area with a normal component that is substantially greater than an associated tangent component.

5. The system of claim 4, wherein the planar coil is incorporated into a wrist band, such that the blood vessel extending through the sensing area is in proximity to the longitudinal axis of the planar coil.

6. The system of claim 4, wherein the planar coil is incorporated into a sensor structure configured for mounting to an arm of a pair of spectacles, such that the planar coil is locatable in proximity to a temporal region of a head.

7. The system of claim 1, wherein:
the resonator control circuitry comprises negative impedance circuitry configured to present to the resonator a negative impedance controlled in response to a negative impedance control signal, so as to maintain the resonator resonance state at the resonant frequency state representative of blood hemoglobin flow through the sensing area; and
the IDC loop circuitry comprises impedance control circuitry configured to determine changes in resonance state relative to such resonant frequency state based on changes in resonator oscillation amplitude, and generate the negative impedance control signal;
the negative impedance circuitry and the impedance control circuitry establishing a negative impedance control loop, including the resonator as a loop filter, operable to control the negative impedance presented to the resonator to counterbalance a resonant impedance of the resonator, thereby maintaining the resonant frequency state;
wherein the output sensor data corresponds to the negative impedance control signal, such that the output sensor data corresponds to the negative impedance required to counterbalance resonator resonant impedance as representative of blood flow through the sensing area.

8. The system of claim 1, wherein the IDC unit further comprises:
resonator frequency circuitry configured to generate a resonator frequency output corresponding to resonator frequency, including resonator frequency for the resonant frequency state, such that the sensor data is provided by at least one of the IDC control loop output and the resonator frequency output.

9. An inductance-to-digital conversion (IDC) circuit operable with a resonant sensor in an inductive sensing system adapted for noninvasive measurement of blood flow through a blood vessel within a body, the resonant sensor adapted for disposition external to the body, in proximity to the blood vessel, the resonant sensor including a resonator with a resonator coil, the resonator characterized by a resonance state (resonator oscillation amplitude and resonator frequency), including a resonant frequency state (steady-state oscillation), the resonator operable to generate, from the resonator coil, a magnetic field within a sensing area that includes the blood vessel, the resonator changing resonance state based on changes in blood flow as characterized by a flow of blood hemoglobin through the sensing area, the IDC circuit comprising:
resonator control circuitry configured to adjust resonator resonance state in response to a resonance control signal; and
IDC loop circuitry configured to determine changes in resonance state relative to a resonant frequency state representative of blood hemoglobin flow through the sensing area, and generate the resonance control signal;
the resonator control circuitry and the IDC loop circuitry establishing an IDC control loop, including the resonator as a loop filter, operable to maintain the resonator resonance state at the resonant frequency state representative of blood hemoglobin flow through the sensing area; and
sensor data output circuitry configured to output sensor data corresponding to the resonance control signal, such that the output sensor data corresponds to the resonant frequency state as representative of blood flow through the sensing area.

10. The IDC circuit of claim 9, wherein:
the resonator control circuitry comprises negative impedance circuitry configured to present to the resonator a negative impedance controlled in response to a negative impedance control signal, so as to maintain the resonator resonance state at a resonant frequency state representative of blood hemoglobin flow through the sensing area; and
the IDC loop circuitry comprises impedance control circuitry configured to determine changes in resonance state relative to such resonant frequency state based on changes in resonator oscillation amplitude, and generate the negative impedance control signal;
the negative impedance circuitry and the impedance control circuitry establishing a negative impedance control loop, including the resonator as a loop filter, operable to control the negative impedance presented to the resonator to counterbalance a resonant impedance of the resonator, thereby maintaining the resonant frequency state;
wherein the output sensor data corresponds to the negative impedance control signal, such that the output sensor data corresponds to the negative impedance required to counterbalance resonator resonant impedance as representative of blood flow through the sensing area.

11. The IDC circuit of claim 9, further comprising:
resonator frequency circuitry configured to generate a resonator frequency output corresponding to resonator frequency, including resonator frequency for the resonant frequency state, such that the sensor data is provided by at least one of the IDC control loop output and the resonator frequency output.

12. The IDC circuit of claim 9, wherein the resonant sensor is configured with an axial coil, such that the blood vessel extends axially within the coil, and such that the sensing area is in the axial region of the axial coil.

13. The IDC circuit of claim 12, wherein the axial coil is incorporated in one of a finger ring in which the blood vessel is within a finger, and a wrist band in which the blood vessel is within a wrist.

14. The IDC circuit of claim 9 wherein the resonant sensor is configured with a planar coil, such that the sensing area is spaced from, and substantially orthogonal to a longitudinal axis of, the planar coil, and the magnetic field within the sensing area is characterized by magnetic field vector magnitudes that intersect the sensing area with a normal component that is substantially greater than an associated tangent component.

15. The IDC circuit of claim 14, wherein the planar coil is incorporated into a wrist band, such that the blood vessel extending through the sensing area is in proximity to the longitudinal axis of the planar coil.

16. The IDC circuit of claim 14, wherein the planar coil is incorporated into a sensor structure configured for mounting to an arm of a pair of spectacles, such that the planar coil is locatable in proximity to a temporal region of a head.

17. A method adaptable for noninvasive measurement of blood flow through a blood vessel within a body, the method operable in an inductive sensing system including a resonant sensor adapted for disposition external to the body, in proximity to the blood vessel, the resonant sensor including a resonator with a resonator coil, the resonator characterized by a resonance state (resonator oscillation amplitude and resonator frequency), including a resonant frequency state (steady-state oscillation), the resonator operable to generate, from the resonator coil, a magnetic field within a sensing area that includes the blood vessel, the resonator changing resonance state based on changes in blood flow as characterized by a flow of blood hemoglobin through the sensing area, the method comprising:
determining changes in resonance state of the resonator relative to a resonant frequency state representative of blood hemoglobin flow through the sensing area, and generating a corresponding resonance control signal; and
adjusting the resonator resonance state in response to the resonance control signal to maintain the resonator resonance state at the resonant frequency state;
such that generating the resonance control signal, and in response, adjusting the resonator resonance state, establishes an IDC control loop, incorporating the resonator as a loop filter, that is operable to maintain the resonator resonance state at the resonant frequency state representative of blood hemoglobin flow through the sensing area; and
outputting sensor data corresponding to the resonance control signal, such that the output sensor data corresponds to the resonant frequency state as representative of blood flow through the sensing area.

18. The method of claim 17, wherein the resonant sensor is configured with an axial coil, such that the blood vessel extends axially within the coil, and such that the sensing area is in the axial region of the axial coil.

19. The method of claim 9 wherein the resonant sensor is configured with a planar coil, such that the sensing area is spaced from, and substantially orthogonal to a longitudinal axis of, the planar coil, and the magnetic field within the sensing area is characterized by magnetic field vector magnitudes that intersect the sensing area with a normal component that is substantially greater than an associated tangent component.

20. The method of claim 17, wherein:
determining changes in resonance state of the resonator is accomplished by determining changes in resonator oscillation amplitude, and generating, as the resonance control signal, a negative impedance control signal;
adjusting the resonator resonance state is accomplished by presenting to the resonator a negative impedance controlled in response to the negative impedance control signal, so as to maintain the resonator resonance state at a resonant frequency state representative of blood hemoglobin flow through the sensing area; and
such that determining changes in resonator oscillation amplitude, and presenting to the resonator a controlled negative impedance, establishes a negative impedance control loop operable to control the negative impedance presented to the resonator to counterbalance a resonant impedance of the resonator, thereby maintaining the resonant frequency state;
wherein the output sensor data corresponds to the negative impedance control signal, and thereby the negative impedance required to counterbalance resonator resonant impedance as representative of blood flow through the sensing area.
